(19) United States
(12) Patent Application Publication
(10) Pub. No.: US 2008/0255649 A1
(43) Pub. Date: Oct. 16, 2008

(54) Electrode Module for Muscle Maintenance and/or Muscle Development

(75) Inventor: Patrick Herbert, Bourguebus (FR)

Correspondence Address: CANTOR COLBURN, LLP, 20 Church Street, 22nd Floor, Hartford, CT, 06103, US

(73) Assignee: SPORT ELEC SA, Bourgtheroulde Infreville (FR)

(21) Appl. No.: 12/090,218

(22) PCT Filed: Oct. 14, 2005
(86) PCT No.: PCT/FR2005/002544

§ 371 Date: Apr. 14, 2008

(51) Int. Cl. A61N 1/22

(52) U.S. Cl. 607/149

(57) ABSTRACT

The invention concerns an electrode module (1) for muscle maintenance and/or muscle development designed to be retained in position by means of an accessory. The invention is characterized in that it comprises a housing delimited by an outer wall (2) attached to an inner wall (9) made of a deformable electrically conductive material and designed to be urged into contact with the region to be treated, said housing including at least one lining block (3) made of a deformable material supported on said outer wall and a conductive end (5) of an electric cable (6) sandwiched between the lining block and the inner wall.

### TECHNICAL FIELD OF THE INVENTION

The present invention relates to an electrode module for muscle maintenance and/or muscle development, which is intended to be held in position with the aid of an accessory such as for example a belt, armband or leg-strap.

### BRIEF DESCRIPTION OF RELATED ART

For this type of application, it is already known to use electrode modules which are adapted to domestic-use generators and which generally comprises a flexible conductive element whose face, intended to come in contact with the user's skin, is covered with a layer made of a material that allows both the passage of an electrical current and adhesion to the skin.

This layer generally comprises a spongy fabric or an echography gel.

An accessory indented to cover a plurality of electrode modules, placed in contact with the skin, may also be employed. Depending on whether the muscles to be trained are the abdominal wall muscles, the biceps, the pectorals, or the quadriceps and the hip-leg muscles, for example, the accessory used will have the form of a belt, an armband, a vest or a leg-strap.

In all cases, the phase during which the electrode modules are put in place remains intricate and laborious. This is because each electrode module has to be dealt with individually, and the application of an intermediate layer used as a connection between the skin and the electrode module must be carried out perfectly in order to avoid any loss of adhesion and in order to ensure correct transmission of the electrical energy.

The particular case of echography gel furthermore entails other drawbacks. This is because such a gel needs to be regenerated by adding water before each use. In practice, a protective sheet covering the gel is removed, then this gel is wetted with water, and it is necessary to wait for complete absorption of the water by the gel before any further action. Furthermore, the gel must keep a constant degree of wetness in order to maintain its adhesive power and a low resistivity. However, the gel has a tendency to dry out in contact with the skin and owing to the heating caused by passage of the electrical current. The increase in the resistivity due to drying of the gel leads to a decrease in the power transmitted by the generator to the skin through the electrode module, for which the user will try to compensate by increasing the power of the generator. This compensation, however, is very unpleasant for the user because disagreeable stinging will very quickly be felt at certain points, which are less “dry” than others and thus favor passage of the current.

### BRIEF SUMMARY OF THE INVENTION

The invention remedies the various drawbacks mentioned above, and to that end it provides an electrode module for muscle maintenance and/or muscle development, which is intended to be held in position with the aid of an accessory such as for example a belt, armband or leg-strap, characterized in that it comprises a compartment delimited by an outer wall, connected to an inner wall which is made of a deformable material that conducts electrical energy and is intended to come in contact with the region to be trained, said compartment containing on the one hand at least one lining pad made of a deformable material resting on said outer wall and, on the other hand, a conductive end of an electrical cable sandwiched between the lining pad and the inner wall.

Thus, when the accessory is correctly placed and is holding the electrode modules according to the invention in position, each of them forms a bulge which protrudes from said accessory owing to the incorporation of a lining pad made of a material which can be deformed in order to adapt optimally to the user's morphology. The contact between each electrode module and the user's skin is therefore perfectly optimized even in the event of movements by the user, and it is then possible to replace the spongy fabric or the echography gel traditionally employed by a simple layer of a cosmetic cream.

It should furthermore be understood clearly that each electrode module may be fixed temporarily or permanently in the corresponding accessory. When the electrode modules are already fixed in the accessory, it is then sufficient for the user to apply a layer of cosmetic cream on the region to be trained, and then to position the accessory correctly around said region. The intricate and laborious step of fastening each electrode module on the skin is therefore obviated.

According to a preferred embodiment of the invention, a double-sided adhesive wall is interposed between the lining pad and the conductive end of the electrical cable.

Preferably, the inner wall is produced in the form of a silicone-carbon plate.

Also preferably, the lining pad is made in the form of a substantially parallelepipedal foam pad.

Advantageously, the conductive end of the electrical cable has the form of conductive filaments arranged in a star-shape.

Also advantageously, the outer wall is produced in the form of a reinforced fabric plate. Furthermore, the outer wall is preferably covered at least partially with a fabric having hooks oriented outward. This then allows the electrode module to be fixed removably on at least one region of the accessory, equipped with a looped fabric, as in the case of Velcro® type fastening. An obvious alternative embodiment comprises the outer wall and the accessory respectively being equipped with a looped fabric and a hooked fabric.

In the context of the present invention, it should be understood clearly that the term “skin” corresponds equally well to the outer layer of the human body and to that of animals.

### DESCRIPTION OF THE DRAWINGS

The invention will be understood more clearly with the aid of the detailed description which is explained below with reference to the appended drawing, in which:

FIG. 1 is an exploded perspective view of an electrode module according to the invention;

FIG. 2 is cross-sectional view of the electrode module according to FIG. 1, once assembled;

FIG. 3 is a perspective view of an electrode module according to the invention with removable fastening;

FIG. 4 is a perspective view of another electrode module according to the invention, fixed permanently on an accessory;

FIG. 5 is a perspective view of a belt equipped with electrode modules corresponding to the one represented in FIG. 4.

### DESCRIPTION OF THE PREFERRED EMBODIMENTS

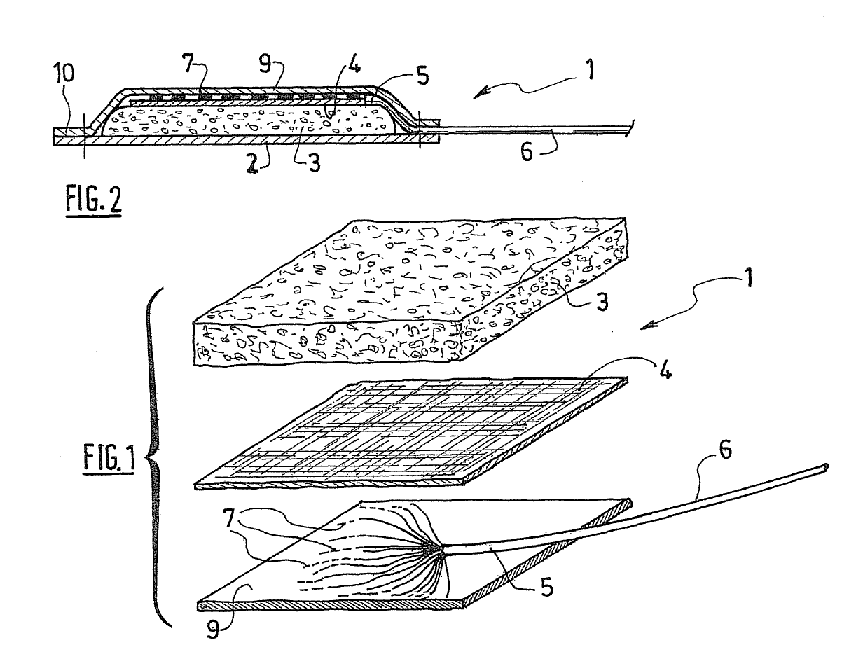

An electrode module 1 according to the invention, as represented in FIGS. 1 and 2, is obtained from an outer wall 2 produced in the form of a reinforced fabric plate (not shown in FIG. 1) with a substantially rectangular shape, made of a material such as 600 D polyester.

A lining pad 3, comprising a parallelepipedal pad of polyurethane foam, is subsequently fitted on the outer wall 2.

A double-sided adhesive wall 4 with a rectangular shape is subsequently applied onto the lining pad 3, and adheres to it by one of its two faces.

A conductive end 5 of an electrical cable 6 is then applied onto the other face of the adhesive wall 4. More precisely, the conductive end 5 has the form of conductive filaments 7 arranged in a star-shape and adhesively bonded flatly onto the adhesive wall 4, and the electrical cable 6 is provided with an opposite second end 8 which is intended to be connected to an electrical energy generator (not shown).

Lastly, an inner wall 9 produced in the form of a substantially rectangular silicone-carbon plate is fitted onto the adhesive wall 4 and adheres to it. This inner wall 9 furthermore has a border 10 fixed, for example by welding, into the border which is on the outer wall 2.

This electrode module 1 formed in this way is then ready to be fixed, either temporarily or permanently, in a corresponding accessory.

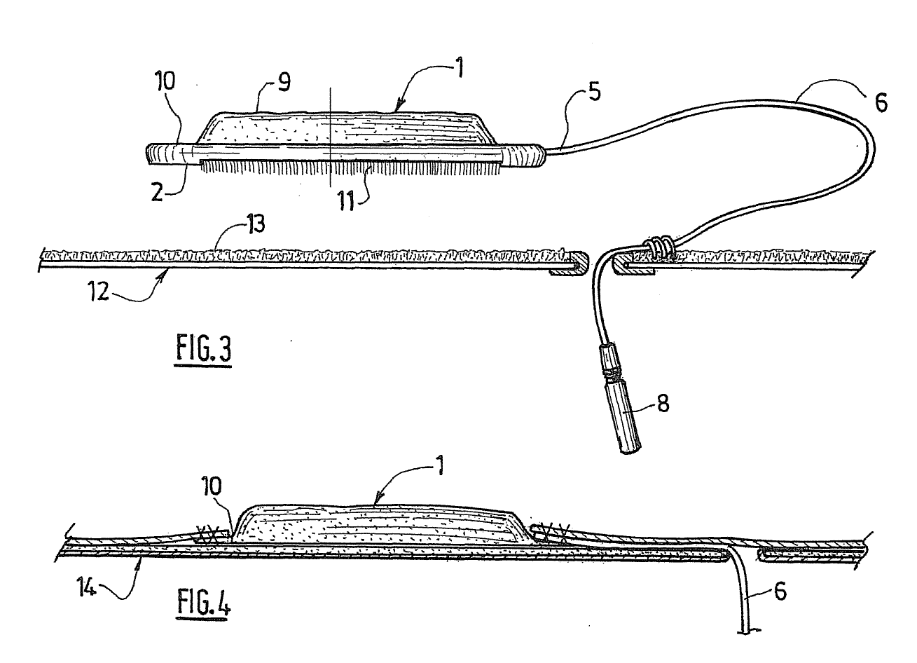

In FIG. 3, the outer wall 2 of the electrode module 1 has been entirely covered with a strip of fabric having hooks oriented outward, so as to allow fastening of the Velcro® type on the inside face of an accessory 12, for example of the belt type, which is covered with a strip of looped fabric 13. The user can thus very easily fix each electrode module 1 removably at the desired positions.

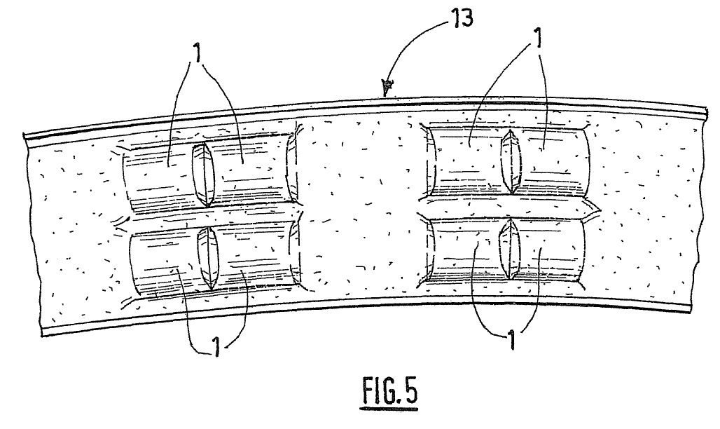

In FIG. 4, conversely, the electrode module 1 is fixed permanently in the inside face of an accessory 14 of the belt type, following a stitching operation carried out in the border 10 of the inner wall 9. The accessory 14 equipped in this way is partially represented in FIG. 5.

As may be deduced from FIGS. 3 and 4, each electrode module 1 then constitutes an inwardly oriented bulge adapted to come perfectly in contact with the user's skin, which makes it possible to optimize passage of the electrical current. The fact that a deformable material is used to make the lining pad 3 is advantageous insofar as this allows the electrode module 1 to adapt as much as possible to the user's morphology.

Furthermore, another advantage in the case of permanent fastening is that it is sufficient to apply a layer of cosmetic cream on the region to be trained, and then to position the accessory 14 correctly. The step of fastening each electrode module 1 on the user's skin is therefore obviated.

Although the invention has been described in connection with particular embodiments, it is very clear that it is not limited in any way thereto and that it comprises all the technical equivalents of the described means as well as combinations of them, so long as they fall within the scope of the invention.

1. An electrode module for muscle maintenance and/or muscle development, which is intended to be held in position with the aid of an accessory such as for example a belt, armband or leg-strap, comprising a compartment delimited by an outer wall, connected to an inner wall which is made of a deformable material that conducts electrical energy and is intended to come in contact with a region to be trained, said compartment containing at least one lining pad made of a deformable material resting on said outer wall and, a conductive end of an electrical cable sandwiched between the lining pad and the inner wall.

2. The electrode module as claimed in claim 1, comprising a double-sided adhesive wall interposed between the lining pad and the conductive end of the electrical cable.

3. The electrode module as claimed in claim 1, wherein the inner wall is produced in the form of a silicone-carbon plate.

4. The electrode module as claimed in claim 1, wherein the lining pad comprises a substantially parallelepipedal foam pad.

5. The electrode module as claimed in claim 1, wherein the conductive end of the electrical cable has the form of conductive filaments arranged in a star-shape.

6. The electrode module Has claimed in claims 1, wherein the outer wall comprises a reinforced fabric plate.

7. The electrode module as claimed in claim 1, wherein the outer wall is covered at least partially with a fabric having hooks oriented outward.

8. An accessory intended to be positioned around a user's body, comprising at least one electrode module as claimed in of claim 1.

9. The accessory as claimed in claim 8, comprising an inside face covered at least partially with at least one strip of looped fabric.
